(19) United States
(12) Patent Application Publication
(10) Pub. No.: US 2012/0183981 A1
(43) Pub. Date: Jul. 19, 2012

(54) METHOED FOR DETECTING CIRCULATING CARTILAGE OLIGOMERIC MATRIX PROTEIN IN THE DIAGNOSIS AND MONITORING OF CIRRHOSIS

(75) Inventors: Gary L. Norman, San Clemente, CA (US); Zakera Shums, San Diego, CA (US)

(73) Assignee: Inova Diagnostics, Inc., San Diego, CA (US)

(21) Appl. No.: 13/392,749

(22) PCT Filed: Aug. 30, 2010
(86) PCT No.: PCT/US10/47159

§ 371 Date: Mar. 30, 2012

Related U.S. Application Data
(60) Provisional application No. 61/238,041, filed on Aug. 28, 2009.

(51) Int. Cl. G01N 33/577

(52) U.S. Cl. 435/7.94; 435/7.92

(57) ABSTRACT

The present invention is in the field of diagnostics, and in particular to the early diagnosis of liver cirrhosis in a patient not known to have liver cancer by detecting and measuring circulating cartilage oligomeric matrix protein (COMP) alone or in conjunction with one or more additional liver cirrhosis biomarkers in the biological fluid of a subject.

### CROSS-REFERENCE TO RELATED APPLICATIONS

This application is a United States national stage application of International Application no. PCT/US10/47159 entitled “Method for Detecting Circulating Cartilage Oligomeric Matrix Protein in the Diagnosis and Monitoring of Liver Cirrhosis” and filed Aug. 30, 2010, which claims priority to U.S. provisional patent application No. 61/238,041 entitled “Method for Detecting Circulating Cartilage Oligomeric Matrix Protein in the Diagnosis and Monitoring of Liver Cirrhosis” and filed Aug. 28, 2009. The contents of the PCT patent application and the United States provisional patent applications are incorporated herein by reference in their entirety as if set forth verbatim.

### TECHNICAL FIELD

The present invention is in the field of diagnostics, and in particular to the early diagnosis of liver cirrhosis in a patient not known to have liver cancer by detecting and measuring circulating cartilage oligomer is matrix protein (COMP) alone or in conjunction with one or more additional liver cirrhosis biomarkers in the biological fluid of a subject.

### BACKGROUND OF THE INVENTION

The liver is the largest organ in the body. Its principal function is removal or neutralization of harmful substances in the blood and the production of immunomodulators. It also plays a role in clotting and absorption.

Cirrhosis and chronic liver failure together were the twelfth most common cause of death in the United States in 2002, accounting for 27,257 deaths. Approximately 40% of patients with cirrhosis are asymptomatic, and the condition is often discovered during a routine examination with laboratory or radiographic studies.

In cirrhosis of the liver, scar tissue replaces normal tissue, which blocks blood flow and prevents the liver from functioning properly. It is most often caused by alcoholism, viral infection, autoimmune disease and genetic defects. The condition is non-reversible with treatment generally focused on reducing progression of the disease. Cirrhosis has also been shown to cause hepatocellular carcinoma, which has an extremely high mortality rate.

Cirrhosis can he diagnosed on the basis of a combination of factors, such as symptoms, detection of circulating serum markers associated with cirrhosis and diagnostic imaging. However, the reference standard for diagnosis requires a biopsy. Circulating serum markers include, for example, aminotransferases (AST and ALT), alkaline phosphatase, bilirubin and albumin. While the specificity and sensitivity of these assays assist in the diagnosis of liver cirrhosis, they are not definitive determining factors, nor are they useful in detecting early stage cirrhosis.

COMP, also known as thrombospondin is a pentameric glycoprotein of the thrombospondin family found primarily in articular cartilage, but also in minute amounts in tendons, synovium, and ligaments. It is a non-cartilage extracellular matrix protein with a mass of 434 kDa. It is not detected in other cartilage-rich structures such as skin and lungs.

When cartilage breaks down, COMP is one of the molecular fragments released into the synovium, and subsequently appears in the blood. In individuals with various forms of arthritis, such as rheumatoid arthritis (RA) and osteoarthritis (OA), the amount of COMP in the serum correlates with the intensity of the disease. Higher concentrations of COMP are seen in individuals with more aggressive cartilage degradation.

COMP is not significantly expressed in normal liver tissue and only rarely (1 of 14) in patients with cirrhosis (Y. Xiao et al., J. Gastroenterol. and Hepatol. 19:296 (2004).) Using an immunohistochemical assay, Xiao et al. demonstrated faint COMP expression in normal liver tissue, moderate expression in cirrhotic liver tissue, and strong expression in hepatic cell carcinoma (HCC) tissue. Connective tissue cells, bile duct cells, and blood vessels showed no detectable expression. Xiao et al, speculated that the few COMP-positive hepatocytes present in liver tissue samples from cirrhotic patients may represent early signs of pre-malignant lesions.

In a similar mRNA expression and immunohistochemical study on normal pancreatic tissue, chronic pancreatitis tissue, and pancreatic cancer tissue, Q, Liao et al, (Rand, J. Gastroenterol, 38:207-215 (2003)) reported preferential expression of COMP in degenerating acinar cells in chronic pancreatitis and chronic pancreatitis-like areas of pancreatic cancer. They speculated that the observed COMP expression was similar to that seen in the early stages of articular cartilage degeneration.

Although the aforementioned studies are useful in determining the pathophysiology of organ disease, diagnostic tests that require a tissue sample are not preferred over less invasive forms of assays that utilize easily obtainable biological fluids such as blood. In addition, there have been no reports that COMP is present in circulation at high enough levels to make it useful as an early diagnostic marker for liver-associated diseases.

Chronic liver injury resulting from viral, immune, or metabolic insults can induce the accumulation of fibrillar extracellular matrix (ECM) components and the development of hepatic fibrosis (D. Manning et at, Gastroenterology 134: 1670-1681 (2008).) In contrast, in the arthritic condition, the ECM is degraded and the fragments make their way into the bloodstream. Accordingly, assays designed to detect circulating COMP would not be expected to he useful in the diagnosis of liver injury where the ECM accumulates in the tissue.

Consequently, there is a need for an improved diagnostic assay for sensitive early detection of liver cirrhosis using circulating marker proteins. In addition, evidence now supports the idea that hepatic fibrosis is not always irreversible (Manning, supra) and therefore circulating COMP detection provides for a useful and convenient means for monitoring development and/or regression of fibrosis.

### SUMMARY OF THE INVENTION

The present invention relates, in part, to a method for evaluating a likelihood that a subject not known to have liver cirrhosis or liver cancer either has undiagnosed liver cirrhosis, or will develop liver cirrhosis, said method comprising the steps of; a) obtaining a blood, serum or plasma sample from the subject; h) mixing the sample with an antibody specific for cartilage oligomeric protein (COMP) to form a COMP-antibody complex; and c) detecting the complex, wherein complex detection indicates said likelihood.

Other aspects of the invention are described throughout the specification.

### DETAILED DESCRIPTION OF THE INVENTION

in the description that follows, a number of terms used in the field of molecular biology, immunology and medicine are extensively utilized. In order to provide a clearer and consistent understanding of the specification and claims, including the scope to be given such terms, the following non-limiting definitions are provided. Unless defined otherwise, all terms used herein have the same meaning as are commonly understood by one of skill in the art to which this invention belongs. In the event that there is a plurality of definitions for a term herein, those in this section shall prevail. All patents, patent applications and publications referred to throughout the disclosure are incorporated herein by reference in their entirety.

When the terms “one,” “a,” or “an” are used in this disclosure, they mean at least one” or “one or more,” unless otherwise indicated.

The term “antibody” as used herein refers to an immunoglobulin molecule which is capable of binding an epitope or antigenic determinant. The term “antibody” includes whole antibodies and antigen-binding fragments thereof, including single-chain antibodies. Such antibodies include human antigen binding antibody and antibody fragments, including, but not limited to, Fab, Fab′ and F(ab′)₂, Fd, single-chain Fvs (scFv), single-chain antibodies, disulfide-linked Fvs (sdFv) and fragments comprising either a V_{L} or V_{H} domain. The antibodies may be from any animal origin including birds and mammals, e.g., human, murine, rabbit, goat, guinea pig, camel, horse and the like.

The term “antigen” as used herein refers to a molecule capable of being bound by an antibody or a T cell receptor (TCR) if presented by MHC molecules. An antigen may be additionally capable of being recognized by the immune system and/or being capable of inducing a humoral immune response and/or cellular immune response leading to the activation of B- and/or T-lymphocytes. An antigen may have one or more epitopes (B- and T-epitopes). Antigens as used herein may also be mixtures of several individual antigens.

The term “antigenic determinant” as used herein refers to a portion of an antigen that is specifically recognized by either B- T-lymphocytes. Antigenic determinants or epitopes are those parts of an antigen that are recognized by antibodies, or in the context of an MHC, by T-cell receptors. An antigenic determinant may contain one or more epitopes.

The term “autoantibody” used. Herein refers to an antibody directed against self-protein, carbohydrate or nucleic acid.

The term “epitope” as used herein refers to a portion of an antigen that is recognized by the immune system, such as an antibody (e,g,, autoantibody), B-lymphoeyte, or T-lymphocyte, and thus the particular domain, region or molecular structure to which the antibody, B-lymphocyte, or T-lymphocyte binds.

The term “portion” as used herein, in reference to a protein, refers to fragments of that protein. The fragments may range in size from two amino acid residues to the entire amino acid sequence minus one amino acid.

The term “variant” or “variants” as used herein refer to a protein or portion thereof which comprises an amino acid sequence having one or more amino acid substitutions, deletions and/or additions (e.g., fusion proteins) as compared to the native amino acid sequence of the protein but which nonetheless retains the proteins immunological activity. These functionally or immunologically equivalent variants may occur as natural biological variations (e,g. polypeptide allelic variants, polypeptide orthologs or splice variants) or they may be prepared using known techniques, such as by chemical synthesis or modification or mutagenesis.

The term “subject” as used herein refers to an animal, including, but limited to, an ovine, bovine, ruminant, lagomorph, porcine, equine, canine, feline, rodent or primate, e.g. a human. Typically, the terms “subject” and “patient” are used interchangeably herein in reference, for example, to a mammalian subject, particularly a human subject.

The term “sample” as used herein refers to biological samples obtained from animals (e.g., humans) and refers to a biological material or compositions found therein, including, but not limited to, bone marrow, blood, serum, cells, plasma, interstitial fluid, urine, cerebrospinal fluid, nucleic acid, DNA, tissue, and purified or filtered forms thereof. Samples for detecting circulating antigens include, for example, blood, serum and plasma.

The term “control” or “control sample” refers to one or more samples, such as a serum sample, taken from at least one subject who has tested negative for the determinant (e.g., antigens or antibodies) in question. For example, a control for testing for the presence of COMP antigen might be taken from a subject who does not have HCC or any other liver disorder that would cause or result in circulating COMP.

The term “diagnosis” as used herein is not meant to imply an absolute diagnosis of a disease such as cirrhosis or cancer, which can normally only be done by performing a biopsy. Instead, it is meant to refer to the determination that a patient is more likely to have the disease than not. As such, detecting the presence of a diagnostic marker such as COMP can be used in conjunction with other diagnostic tests to help confirm a diagnosis, or to monitor treatment. In addition, the term “diagnosis” can mean that a patient who is not known to have a disease, whether or not a biopsy has been performed, is more likely than not to develop the disease, or is more likely than not to actually have the disease in the absence of a positive biopsy. Accordingly, a marker is “diagnostic” if its detection is positively correlated with the presence of or the likelihood of developing a disease of interest.

The present invention relates, in part, to a method for evaluating a likelihood that a subject not known to have liver cirrhosis or liver cancer either has undiagnosed liver cirrhosis, or will develop liver cirrhosis. The method involves obtaining a blood, serum or plasma sample from the subject, mixing the sample with an antibody specific for COMP to form a COMP-antibody complex, and detecting the complex, wherein complex detection indicates such a likelihood.

### COMP

Cartilage Oligomeric Matrix Protein (COMP) is a member of the thrombospondin family of proteins and is a pentameric extracellular matrix protein with a subunit size of 95-97 kDa. It has been observed to he localized in the extracellular matrix of chondrocytes, synovium, tendons and ligaments. Increased levels of COMP have been reported in tissues from patients with rheumatoid arthritis and hepatocellular carcinoma (HCC) (Xiao, Y. et al. J. Gastroenterol, and Hepatol. 19:296-302, 2004). In this publication the authors found that while “COMP was absent or rarely expressed in the normal liver and liver cirrhosis tissues . . . ” it was “significantly over-expressed in HCC tissue”. In view of these findings it would have been unobvious to attempt to utilize COMP as a marker for diagnosis of liver cirrhosis. However, contrary to Xiao et al., the present invention is based on the finding that unexpectedly high concentrations of COMP exist consistently in the serum from patients suffering from liver cirrhosis. This observation lead to the development of the present assay method for the diagnosis of liver cirrhosis based on the detection of elevated COMP levels.

### COMP Antibodies

In a number of assay formats and particularly for an enzyme-linked immunosorbent assay (ELISA), detection of COMP usually involves the preparation of two antibodies that bind to two separate epitopes present on COMP. Preferably, the antibodies are monoclonal antibodies with high sensitivity and affinity for specific epitopes of the protein.

Antibodies employed to achieve high sensitivity in diagnostic immunoassays should have both high affinity for the antigen and good binding kinetics. As optimal interactions of monoclonal antibodies with antigens are important to achieve required levels of sensitivity, methods by which these antibodies are selected are also important. It has been observed that the bond formed between an antibody and its corresponding antigen can result in the antigen having undergone a conformational change or stoichiometric rearrangement. This conformational change can greatly affect the ability of a second antibody to bind to the antigen. This stoichiometric hindrance or enhancement, when it occurs, can significantly affect the success or failure of the assay sensitivity (Johne B., et al., J. Immunol. Methods, 1993, 160:191-198). Because of this, the monoclonal antibodies of the present invention are screened to assure that they retain their binding selectivity and sensitivity for their epitope, particularly when if the protein of interest is bound by another antibody. Such screening methods and ultimately the selection of antibody pairs are easily carried out using routine optimization experiments.

The monoclonal antibodies of the present invention are prepared by conventional methods, generally following the protocols of Kohlers and Milstein (Nature, 256, 495-497, 1975; Eur. J, Immunol. 6, 511-519, 1976). According to this method, tissue culture adapted mouse myeloma cells are fused to antibody producing cells from immunized mice to obtain the hybrid cells that produce large amounts of a single antibody molecule. In general, the antibody producing cells are prepared by immunizing an animal, for example, mouse, rat, rabbit, sheep, horse, or bovine, with an antigen. The immunization schedule and the concentration of the antigen in suspension is such as to provide useful quantities of suitably primed antibody producing cells. These antibody producing cells can be either spleen cells, thymocytes, lymph node cells and/or peripheral blood lymphocytes.

The antibody producing cells are then fused with myeloma cells, cell lines originating from various animals such as mice, rats, rabbits, and humans, can be used, using a suitable fusion promoter. Many mouse myeloma cell lines are known and available generally from members of the academic community and various depositories, such us the American Type Culture Collection, Rockville, Md. The myeloma cell line used should preferably be medium sensitive so that unfused myeloma cells will not survive in a selective media, while hybrids will survive. The cell line most commonly used is an 8-azaguanine resistant cell line, which lacks the enzyme hypozarithine-guanine-phosphoribosyol-transferase and therefore will not be supported by hypoxanthine-aminopterin-thymidine (HAT) medium. In general, the cell line is also preferably a “non-secretor” type, in that it does not produce any antibody. The preferred fusion promoter is polyethyleneglycol having an average molecular weight from about 1000 to about 4000. Other fusion promoters such as polyvinylalcohol, a virus or an electrical field can also be used,

The immortalized cells (i.e., hybridomas) must then be screened for those which secrete antibody of the correct specificity. The initial screening is generally conducted using an ELISA. Specifically, the hybridoma culture supernatants are added to microtitre plates which have been previously coated with the antigen. A bound specific antibody (i,e, mouse antibody directed against the antigen) from the culture supernatants can be detected using a labeled second antibody, for example, goat anti-mouse IgG labeled with peroxidase (commercially available). Cultures that are positive against the antigen are then subjected to cloning by the limiting dilution method. Secondary hybridoma cultures are re-screened as described above, and further positive cultures are then examined using the BIAcore system (Pharmacia Biosensor AB, Uppsala, Sweden). The cultures are then evaluated to determine whether or not the antibody produced binds the antigen and the kinetic profile of antigen binding. A selected culture based on these results is subject to further cloning until culture stability and clonality are obtained. Immediately after hybridization, the fusion products will have approximately SO chromosomes, and as these cells proceed to divide they will randomly lose some of these chromosomes. The cloning process provides a method to select those cells which still have the chromosomes coding for antibody production. The cloning process is repeated until 100% of the subpopulation exhibits the production of a specific antibody, which is indicative of the “stability” of the hybridoma. In addition, hybridoma culture wells often have multiple colonies some of which may be antibody non-producers. The cloning process allows the selection of a positive hybrid which is derived from a single cell. This process is repeated for production of monoclonal antibodies directed to specific epitopic regions of a protein desired by providing the specific antigenic determinants for each epitope. The monoclonal antibodies are screened to assure that both are capable of binding the desired antigen simultaneously. This can be done, for example in the case of COMP, by binding one of the monoclonal antibodies to the well of a microtiter plate then adding COMP followed by the addition of the other antibody labeled with a reporter group. By standard methods such as these, monoclonal antibodies that are highly selective and specific for their epitopes can he identified for sandwich assays like the ELISA.

The monoclonal antibody of the present invention can be produced either using a bioreactor or from ascites. Such procedures are well known in the art.

There are several methods of evaluating and analyzing antibody-antigen interactions. The most commonly used and accepted methods include the half sandwich ELISA, full sandwich ELISA, immunoblot and surface plasmon resonance (SPR). In SPR biological reactants (e.g. antibodies or antigens) are covalently attached to a dextran matrix lying on the surface of a gold/glass sensor chip interface. Near infrared light, directed onto the opposite side of the sensor chip surface, is reflected, and also induces an evanescent wave in the gold film, which in turn causes an intensity dip in the reflected light at a particular angle known as the resonance angle. If the refractive index of the sensor chip surface is altered (e.g. by antigen binding to antibody) a shift occurs in the resonance angle. This angle shift can be measured and is expressed as resonance units (RUs) such that 1000 RUs is equivalent to a change in surface protein concentration of 1 ng/mm². These changes are recorded on a sensorgram which depicts the association and dissociation of any biological reaction. In this case, the binding of antibodies to their antigens.

In one embodiment, the assay utilized to detect COMP is a full sandwich ELISA. As mentioned above, this type of assay utilizes a double antibody method for detecting the presence of an analyte. One antibody bond to a solid support (i.e. capture antibody) and the other labeled with a detectible reporter group (i.e. detector antibody) bind different epitopes of the target molecule, in essence, sandwiching the molecule between the two antibodies. These techniques are reviewed in “Rasie Principals of Antigen-Antibody Reaction”, Elvin A. Labat, (Methods in Enzymology, 70: 3-70 (1980)). Such assays are often referred to as fast format systems because they are adapted for rapid determinations of the presence of an analyte. COMP present in a sample of biological fluid is sandwiched between the detector antibody containing a detectable label or reporter group and the capture antibody irreversibly immobilized onto a solid support.

### Diagnostic Assay

There are many different types of immunoassays suitable for use in the present invention. Any of the well known immunoassays may be adapted to detect the level of COMP antigen utilizing an antibody that specifically binds COMP present in a sample, such as, e.g., ELISA, fluorescent immunosorbent assay (HA), chemical linked immunosorbent assay (CLIA), radioimmuno assay (RIA), immunoblotting, gel diffusion precipitation reactions, immunodiffusion assays, in situ immunoassays (e.g., using colloidal gold, enzyme or radioisotope labels, for example). Western blots, precipitation reactions, agglutination assays (e.g., gel agglutination assays, hemagglutination assays, etc.), complement fixation assays, immunofluorescence assays, protein A assays, and immunoelectrophoresis assays, etc. Chapter two of The Immunoassay Handbook, (David Wild, ed., Stockton Press, New York, 1994) provides a comprehensive review of these immunoassays. A competitive immunoassay with solid phase separation or an immunometric assay for antigen detection is particularly suitable for use in the present invention.

In one exemplary embodiment of the invention, the diagnostic assay is an immunometric assay for detecting the level of COMP in a sample. In the immunometric assay, a first COMP antibody is immobilized on a solid support directly or indirectly through a capture agent, such as an anti-immunoglobulin antibody. An aliquot of a sample, such as a serum sample, from a subject is added to the solid support and allowed to incubate with the antibody on the solid phase. A secondary antibody that recognizes a different epitope than the first antibody, or one that recognizes only the antibody-antigen complex is added. After separating the solid support from the liquid phase, the support phase is examined for a detectable signal. The presence of the signal on the solid support indicates that COMP was present in the sample increased optical density or radiolabeled signal when compared to the control samples from normal subjects correlates with a diagnosis of cirrhosis in a subject.

Solid supports are known to those skilled in the art and include the walls of wells of a reaction tray (e.g., microtiter plates), test tubes, polystyrene beads, magnetic beads, nitrocellulose strips, membranes, microparticles such as latex particles, glass or silicon chips, sheep (or other animal) red blood cells, duracytes and others. Suitable methods for immobilizing proteins on solid phases include ionic, hydrophobic, covalent interactions and the like. A solid support, as used herein, refers to any material which is insoluble, or can be made insoluble by a subsequent reaction. The solid support can be chosen for its intrinsic ability to attract and immobilize the protein or antigen also referred to as the capture reagent. Alternatively, the solid phase can retain an additional molecule which has the ability to attract and immobilize the capture reagent. The additional molecule can include a charged substance that is oppositely charged with respect to the capture reagent itself or to a charged substance conjugated to the capture reagent. Alternatively, the molecule can be any specific binding member which is immobilized upon (attached to) the solid support and which has the ability to immobilize the antigen through a specific binding reaction. The molecule enables the indirect binding of the antigen to a solid support material before the performance of the assay or during the performance of the assay.

The signal producing system is made up of one or more components, at least one of which is a reporter group or label, that generate a detectable signal that relates to the amount of bound and/or unbound label, i.e., the amount of label bound or unbound to the COMP-antibody complex. The label is a molecule that produces, or which may be induced to produce, a signal. Examples of labels include fluorophores, enzymes, chemiluminescent molecules, photosensitizers suspendable particles. The signal is detected and may be measured by detecting enzyme activity, luminescence or light absorbance. Radiolabels may also be used and levels of radioactivity detected and measured using a scintillation counter.

Examples of enzymes which may be used to label the anti-immunoglobulin antibody include β-D-galactosidase, horseradish peroxidase, alkaline phosphatase, and glucose-6-phosphate dehydrogenase (“G6PDH”). Examples of fluorescers which may be used to label the anti-human immunoglobulin include fluorescein, isothiocyanate, rhodamines, phycoerythrin, phycocyanin, allophycocyanin, o-phthaldehyde, fluorescamine, and Alexa Fluor® dyes (that is, sulfonated courmarin, rhodamine, xanthene, and cyanine dyes). Chemiluminescent molecules that may be used in the present invention include e.g., isoluminol. For example, the anti-immunoglobulin antibody may be enzyme labeled with either horseradish peroxidase or alkaline phosphatase.

Biotin labeled antibodies may be used as an alternative to enzyme linked antibodies. In such cases, bound antibody would be detected using commercially available streptavidin-horseradish peroxidase detection systems.

Enzyme labeled antibodies produce different signal sources, depending on the substrate. Signal generation involves the addition of substrate to the reaction mixture. Common peroxidase substrates include ABTS (2,2′-azinobis(ethylbenzothiazoline-6-sulfonate)), OPD (O-phenylenediamine) and TMB (3,3′,5,5′-tetramethylbenzidine). These substrates require the presence of hydrogen peroxide. p-Nitrophenyl phospate is a commonly used alkaline phosphatase substrate. During an incubation period, the enzyme gradually converts a proportion of the substrate to its end product. At the end of the incubation period, a stopping reagent is added which stops enzyme activity. Signal strength is determined by measuring optical density, usually via spectrophotometer.

Alkaline phosphatase labeled antibodies may also be measured by fluorometry. Thus in the immunoassays of the present invention, the substrate 4-methylumbelliferyl phosphate (4-UMP) may be used. Alkaline phosphatase dephosphorylated 4-UMP to form 4-methylumbelliferone (4-MU), the fluorophore. Incident light is at 365 nm and emitted light is at 448 nm.

The amount of color, fluorescence, luminescence, or radioactivity present in the reaction (depending on the signal producing system used) is proportionate to the amount of antigen in a sample. Quantification of optical density may be performed using spectrophotometric fluorometric methods, including flow cytometers. Quantification of radiolabel signal may be performed using scintillation counting.

In some embodiments, an automated detection assay is utilized. Methods for the automation of immunoassays include those described in U.S. Pat. Nos. 5,885,530, 4,981,785, 6,159,750, and 5,358,691, each of which is herein incorporated by reference. In some embodiments, the analysis and presentation of results is also automated. For example, in some embodiments, software that generates a prognosis based on the presence or absence of a series of proteins corresponding to disease markers is utilized.

In some embodiments, the COMP level may be used together with other biological markers as a panel for liver cirrhosis diagnosis. The panel allows for the simultaneous analysis of multiple markers correlating with liver disease. For example, a panel may include markers identified as correlating with HBV, HCV or cirrhosis in a subject that is likely, or not likely, to respond to a given treatment. Depending on the subject, panels may be analyzed alone or in combination in order to provide the best possible diagnosis and prognosis. Markers for inclusion on a panel are selected by screening for their presence and predictive value using any suitable method, including but not limited to, those described elsewhere herein,

Assays for detecting COMP are commercially available. However the present invention provides a new use for such assays and the performance of such assays to detect circulating COMP as opposed to tissue associated COMP, as well as the performance of such assays on a particular patient population (i.e. patients not known to have liver cancer) for a particular purpose (i.e., to assess the likelihood that the patient already has liver cirrhosis or will develop it in the future.) Such new use is entirely unexpected, since COMP levels in cirrhotic liver tissues have previously been reported to be very low if observed at all.

### Data Analysis

In the present invention, a computer-based analysis program may also be used to translate the raw data generated by the detection assay into data of predictive value for a clinician. The clinician can readily access the predictive data using any suitable means. The clinician is then able to immediately utilize the information in order to optimize the care of the subject.

The present invention contemplates any method capable of receiving, processing, and transmitting the information to and from laboratories conducting the assays, information provides, medical personal, and subjects. For example, in some embodiments of the present invention, a sample (e.g., a biopsy or a serum or urine sample) is obtained from a subject and submitted to a profiling service (e.g., clinical lab at a medical facility, genomic profiling business, etc.), located in any part of the world (e.g., in a country different than the country where the subject resides or where the information is ultimately used) to generate raw data. Where the sample comprises a tissue or other biological sample, the subject may visit a medical center to have the sample obtained and sent to the profiling center, or subjects may collect the sample themselves (e.g., a urine sample) and directly send it to a profiling center. Where the sample comprises previously determined biological information, the information may be directly sent to the profiling service by the subject (e.g., an information card containing the information may be scanned by a computer and the data transmitted to a computer of the profiling center using an electronic communication system). Once received by the profiling service, the sample is processed and a profile is produced, specific for the diagnostic or prognostic information desired for the subject.

The profile data is then prepared in a format suitable for interpretation by a treating clinician. For example, rather than providing raw expression data, the prepared format may represent a diagnosis or risk assessment (e.g., likelihood of a liver disease such as liver cirrhosis to respond to a specific therapy) for the subject, along with recommendations for particular treatment options. The data may be displayed to the clinician by any suitable method. For example, in some embodiments, the profiling service generates a report that can be printed for the clinician (e.g., at the point of care) or displayed to the clinician on a computer monitor.

In some embodiments, the information is first analyzed at the point of care or at a regional facility. The raw data is then sent to a central processing facility for further analysis and/or to convert the raw data to information useful for a clinician or patient. The central processing facility provides the advantage of privacy (all data is stored in a central facility with uniform security protocols), speed, and uniformity of data analysis. The central processing facility can then control the fate of the data following treatment of the subject. For example, using an electronic communication system, the central facility can provide data, to the clinician, the subject, or researchers.

In some embodiments, the subject is able to directly access the data using the electronic communication system. The subject may chose further intervention or counseling based on the results. In some embodiments, the data is used for research use. For example, the data may be used to further optimize the inclusion or elimination of markers as useful indicators of a particular condition or severity of disease.

### Panel Assays

In addition to measuring the presence of COMP alone, the present invention also contemplates “panel assays” measuring COMP along with one or more other analytes, such as GP73 antigen or autoantibodies. Such panel assays may be provided as a kit containing two or more separate assay platforms or they may be combined on a single platform such as an immunoassay strip or ELISA plate.

### EXAMPLE

### Enzyme Linked immunosorbent Assay (ELISA) For COMP

The COMP antigen present in patient's sera was detected using the solid-phase sandwich immunoassay manufactured by Anamar Medical AB, Uppsala, Sweden. This assay is described in Patent No. 2007/0154969. The assay was performed as specified by the product direction insert. The assay utilizes two monoclonal antibodies directed against two different antigenic determinants present in the COMP molecule. To detect COMP using this assay, one anti-COMP monoclonal antibody is first bound to the wells of a microtiter plate. A sample (or standard of known COMP concentration) to be assayed for the presence of COMP is first diluted 1:10 in sample diluent and 25 ul of the diluted sample is added to the well together with 100 ul of conjugate solution containing a second anti-COMP monoclonal antibody conjugated to horseradish peroxidase. Any COMP present in the sample solution will bind to the anti-COMP antibody attached to the microliter plate well and also to the horseradish peroxidase conjugated anti-COMP antibody in the conjugate solution. After a 2 hour incubation period, the well is aspirated and washed 5-6 times with wash buffer to remove unbound reagents. Bound conjugated anti-COMP antibody is detected by the addition of TMB (3,3′,5,5″tetramethylbenzidine) substrate which produces a yellow color in the presence of horseradish peroxidase. The enzymatic reaction is stopped after 15 minutes by adding a solution of sulfuric acid. The color in the well is read by a spectrophometer at 450 nm. The concentration of COMP in the sample is determined by comparison to a standard curve constructed with absorbance values obtained with standards of known COMP concentrations.

Using the ELISA assay described above, COMP was measured in 187 patients with chronic liver diseases. The 187 patients included 72 with chronic hepatitis B viral (HBV) infection; 75 with chronic hepatitis C viral (HCV) infection, 22 with primary biliary cirrhosis (PBC); 7 with autoimmune hepatitis (AIH) type 1; and 11 with alcoholic liver disease (ALD). Eighty-seven patients had documented cirrhosis and 100 patients had no evidence of cirrhosis. Among the 87 patients with cirrhosis, 12 had developed HCC at the time of serum collection. One hundred and fifty patients were followed up for at least 12 months.

Of the 187 patients, 52 were positive for serum COMP as shown below in Table 1. The overall mean frequency of serum COMP (>15 units) in the various types of chronic liver disease was 27.8%.

TABLE 1
The Frequency of Positive COMP values
(>15 units) in Various Clinical Groups.

| | | COMP > 15 | |
|---|---|---|---|
| DISEASE | NO. | UNITS | |
| AIH | 7 | 2 | (28.6%) |
| PBC | 22 | 5 | (22.7%) |
| HCV | 75 | 19 | (25.3%) |
| HBV | 72 | 20 | (30.6%) |
| ALD | 11 | 4 | (36.4%) |
| TOTAL | 187 | 52 | (27.8%) |

### A. COMP is a Prognostic Marker for Hepatic Cell Carcinoma

Of the 52 COMP positive specimens, 7 (13.5%) had a diagnosis of hepatic cell carcinoma (HCC) at the beginning of the study. Of the 135 COMP negative patients, only 5 (3.7%) had a diagnosis of HCC. The sensitivity of the COMP assay for detecting the 12 patients with a known diagnosis of HCC at the time of serum collection was therefore 58% (7 of 12). These results indicate that COMP is a marker of HCC in chronic liver disease patients.

As described above, COMP was positive in 25.7% (45/175) of patients without a diagnosis of HCC at the beginning of the study. Based on the lack of clinical diagnosis HCC in these 175 patients at the beginning of the study, the specificity of the COMP assay for the diagnosis of HCC was calculated to he 74.3% (130/175). This is an underestimation of the true prognostic specificity, since some of the COMP negative HCC patients developed COMP during follow-up.

One hundred and forty patients without HCC at the beginning of the study (34 patients COMP positive and 106 COMP negative) were followed for 12 months or more. Of the 34 patients positive for COMP at the beginning of the study, but without a diagnosis of HCC, 41.2% (14/34) developed HCC during the follow-up period. In contrast, only 5 of the 106 (4.7%) COMP negative patients developed HCC during the follow-up period.

These results indicate that the measurement of COMP in patients provides a prognostic, non-invasive marker than can precede the clinical recognition of HCC and facilitate early intervention and treatment. Of the 19 patients who developed HCC during follow-up, 73.7% (14/19) were positive for serum COMP prior to the diagnosis of HCC. This is of critical clinical importance, since early diagnosis of HCC is extremely difficult and prognosis for HCC is generally grim. Markers to assist in earlier identification of patients at high risk of developing HCC will result in more intensive and focused monitoring of these patients and detection of DCC at early stages when therapeutic options are most effective.

The finding that 14 of the 19 patients who developed HCC on follow-up were COMP positive, means that the sensitivity of the assay for patients with HCC, or whom developed DCC on follow-up, was 67.7%.

### B. COMP is a Prognostic Marker for Cirrhosis

Assessment of hepatic fibrosis is critical for patient staging, monitoring, and treatment choices. Identification of the presence of cirrhosis or, more importantly, monitoring and identification of the development of cirrhosis is critically important for patients with chronic liver disease, since almost all patients who develop DCC are cirrhotic. Although liver biopsy is considered the “gold standard,” limitations of the procedure include sampling error, inter-observer differences in interpretation, particularly underestimation of disease, high cost, and significant risks of the procedure itself. Hepatic fibrosis evolves to cirrhosis. Since, cirrhosis is generally progressive and reflective of deterioration of the liver, monitoring for its development is of critical clinical significance. Detection of individuals that are of increased risk for progressing to cirrhosis or that have progressed to cirrhosis, but have not yet been diagnosed, is of high clinical value. Non-invasive tests are highly desirable for cost reasons, compliance, and safety reasons.

Examination of the overall frequency of COMP among the 187 patients with and without cirrhosis showed that COMP was present at >15 units in the serum of cirrhotic patients almost 4 times more frequently than in the serum of non-cirrhotic patients (46% v 12%).

Examination of the frequency of COMP among patients with and without cirrhosis stratified by their specific disease diagnosis showed that for the 75 patients with HCV infection, COMP was present almost 10 times more frequently in cirrhotic HCV patients compared to non-cirrhotic patients (48.6% compared to 5%). Similarly, 53.3% of the HBV patients with cirrhosis were positive for COMP, compared to only 14.3% for HEY patients without evidence of cirrhosis. These results are shown below in Table 2.

TABLE 2
Frequency of COMP in the Overall Cohort and in Specific
Disease Groups stratified by Presence of Cirrhosis

| | Overall | | | CIRRHOTIC | | | NON-CIRRHOTIC | | |
|---|---|---|---|---|---|---|---|---|---|
| | | COMP | COMP | Cirrhotic | COMP | COMP | NON- | COMP | COMP |
| Disease | N | Pos # | Pos % | # | Pos # | Pos % | Cirrhotic # | Pos # | Pos % |
| AIH | 7 | 2 | 28.6% | 7 | 2 | 28.6% | 0 | 0 | 0.0% |
| PBC | 22 | 5 | 22.7% | 5 | 2 | 40.0% | 17 | 3 | 17.6% |
| HCV | 75 | 19 | 25.3% | 35 | 17 | 48.6% | 40 | 2 | 5.0% |
| HBV | 72 | 22 | 30.6% | 30 | 16 | 53.3% | 42 | 6 | 14.3% |
| Alcoholic | 11 | 4 | 36.4% | 10 | 3 | 30.0% | 1 | 1* | 100.0% |
| Liver Dis. | | | | | | | | | |
| Total | 187 | 52 | 27.8% | 87 | 40 | 46.0% | 100 | 12 | 12.0% |

*This COMP positive, non-cirrhotic individual developed cirrhosis during the follow-up period.

The demonstration that COMP is most sensitive for detecting cirrhosis in individuals with HBV or HCV infections is of critical significance since about 2 billion people worldwide are estimated to be infected by HBV and 350 million have chronic HBV infection. In China, 8-10% of the adult population is estimated to be chronically infected with HBV. About 25% of those with chronic HBV infection will die from cirrhosis or liver cancer (World Health Organization Fact Sheet No 204, August 2008). Approximately 170 million people worldwide are estimated to be infected with HCV. Chronic infection develops in 80% of those infected with HCV and 1-5% of those with chronic infection progress to liver cancer (World Health Organization Fact Sheet No 164, October 2000).

The sheer number of individuals infected with HBV and HCV make assessment of cirrhosis by imaging or biopsy impractical. In comparison, measurement of serum COMP levels is non-invasive, relatively inexpensive, and can be applied to screen and monitoring large numbers of individuals.

The significance and utility of the measurement of COMP in patients at risk of progressing to cirrhosis is further supported by analysis of the number of COMP positive sera that were found to be from patients with cirrhosis. Of the 187 patients studied, 52 were found to be COMP positive. Almost 77% (76.9%; 40/52) of the COMP positive sera were found to be from patients with cirrhosis (Table 3 below). When the individual disease groups were tested, a similar result was obtained. For HCV patients, 89.5% (17/19) of the specimens that were positive for serum COMP were from HCV patients with cirrhosis. The detection of COMP in patients with chronic liver disease is therefore clearly an important non-invasive marker for detection of cirrhosis as well as surveillance monitoring for the development of cirrhosis in these at-risk patients.

TABLE 3
Most COMP Positive Sera from the Various Chronic Liver Disease
Clinical Groups was from Patients with Cirrhosis

| | | Number of COMP | % COMP positive |
|---|---|---|---|
| | Total number | positive sera which | sera which were |
| | of COMP | were from patients | from patients |
| | positive sera | with cirrhosis | with cirrhosis |
| HBV | 22 | 16 | 72.7% |
| HCV | 19 | 17 | 89.5% |
| Alcoholic | 4 | 3 | 75% |
| Liver Disease | | | |
| AIH | 2 | 2 | 100% |
| PBC | 5 | 2 | 40% |
| Total | 52 | 40 | 76.9% |

In addition to assessing the presence and monitoring the development of cirrhosis. COMP may also have use in monitoring the effectiveness of treatments to slow and cause regression of cirrhosis.

Furthermore, the presence of COMP indicates an increased risk of liver-related death. Of the 150 patients followed, 35% (14/40) of the COMP positive patients suffered a liver-related death in contrast to 17.3% (39/110) of the COMP negative patients. Overall, out of the total of 33 liver-related deaths, 42.4% (14/33) were positive for serum COMP at the beginning of the study.

### C. COMP in HBV Patients

In HBV infected individuals, COMP measurement is also useful to detect cirrhosis or the propensity to develop cirrhosis. In the 72 patients with HBV, 30 were cirrhotic and 42 were non-cirrhotic. 53.3% of the cirrhotic patients were COMP positive compared to only 12% of the non-cirrhotic patients.

In addition, Almost one third of the cirrhotic HBV patients found to be COMP positive (31.25%, 5/16) had a diagnosis of HCC. One non-cirrhotic HBV patient found to he COMP positive was also found to have diagnosed HCC. While about 53% of the cirrhotic HBV patients were positive for COMP, 73% (16/22) of the specimens testing positive for serum COMP were from individuals with cirrhosis. Five individuals without cirrhosis and COMP negative when their sera was collected subsequently developed cirrhosis during the follow-up period. Serum was not available from these individuals during the follow-up period. However, it is likely that COMP would have been detectable during the evolving cirrhosis.

Approximately 73% (8/11) of the cirrhotic HBV patients without a diagnosis of HCC when the serum sample was collected, but whose sera tested positive for COMP, developed HCC during the follow-up period. A striking 81.25% (13/16) of cirrhotic HBV patients with positive COMP results had HCC at the time of sera collection or developed HCC during the follow-period. In addition, Cirrhotic HBV patients testing positive for COMP died more frequently during the follow-up period (43.8%) than cirrhotic COMP negative (28.6%) or non-cirrhotic (2.3%) HBV infected individuals.

### COMP in HCV Patients

Of the 75 patients with HCV, 35 were cirrhotic and 40 were non-cirrhotic. 48.6% of the cirrhotic patients were COMP positive as compared with only 5% of the non-cirrhotic patients. While about 49% of the cirrhotic HCV patients were positive for COMP, almost 90% (89.5%,17/19) of the specimens testing positive for serum COMP were from HCV patients with cirrhosis.

Almost one quarter (23.5%, 4/17) of COMP positive cirrhotic HCV patients had HCC or developed HCC during the follow-up period. In comparison, of 18 HCV patients with cirrhosis but who were negative for serum COMP, only 1 of the 18 (5.5%) had HCC. Three of the 16 (18,8%) COMP positive cirrhotic HCV patients who did not have HCC at the time of sera collection subsequently developed HCC during the follow-up period.

Three of the 1.7 (17.6%) COMP positive cirrhotic HCV patients died compared to 11.1% (2/18) of 18 cirrhotic COMP negative HCV patients. Neither of the two COMP positive non-cirrhotic patients died.

The value of testing several parameters and using the combined results to assess the patients was also examined. It was found that while 14% of the cirrhotic patients were positive for GP73 autoantibody, GP73 antigen, and COMP, none of the non-cirrhotic patients were positive for all 3 assays. Conversely, it was found that while only 4% of the cirrhotic patients were negative for all three assays, 30% of the non-cirrhotic patients were negative for all 3 assays. In addition, 39 healthy controls were negative by both the GP73 autoantibody and GP73 antigen assays.

The examples set forth above are provided to give those of ordinary skill in the art with a complete disclosure and description of how to make and use the preferred embodiments of the present invention, and are not intended to limit the scope of what the inventors regard as their invention. Modifications of the above-described modes for carrying out the invention that are obvious to persons of skill in the art are intended to be within the scope of the following claims. All publications, patents, and patent applications cited in this specification are incorporated herein by reference as if each such publication, patent or patent application were specifically and individually indicated to be incorporated herein by reference.

We claim:

1. A method for evaluating a likelihood that a subject not known to have liver cirrhosis or liver cancer either has undiagnosed liver cirrhosis, or will develop liver cirrhosis, said method comprising the steps of:
a) obtaining a blood, serum or plasma sample from the subject;
b) mixing the sample with an antibody specific for cartilage oligomeric protein (COMP) to form a COMP-antibody complex; and
c) detecting the complex, wherein complex detection indicates said likelihood.

2. The method according to claim 1, wherein the antibody is a monoclonal antibody.

3. The method according to claim 1, wherein the complex is detected in an enzyme-linked immunosorbent assay (ELISA).

4. The method according to claim 1, wherein the antibody specific for COMP is attached to a solid phase.

5. The method according to claim 4 further comprising the step of contacting the complex with a labeled antibody specific for COMP, wherein the antibody specific for COMP attached to a solid phase and the labeled antibody bind to different epitopes on the COMP.
